(19) United States
(12) Patent Application Publication
(10) Pub. No.: US 2002/0058647 A1
(43) Pub. Date: May 16, 2002

(54) Pamidronate solution

(76) Inventors: Gregory Paul Handreck, Victoria (AU); Wei Zhou, Victoria (AU); Russell John Tait, Victoria (AU)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 09/773,480

(22) Filed: Feb. 2, 2001

(30) Foreign Application Priority Data
Sep. 18, 2000 (AU) PR 0189

(51) Int. Cl. A61K 31/66

(52) U.S. Cl. 514/102

(57) ABSTRACT

A stable injectable solution containing pamidronate and a method for preparing a therapeutic aqueous disodium pamidronate solution. The method comprises preparing a slurry of pamidronic acid in water, combining aqueous sodium hydroxide with the slurry in an amount about 2:1 molar ratio of sodium hydroxide to pamidronic acid to yield a solution of disodium pamidronate having visual clarity and a pH of about 6.5, and packaging the solution in sealed containers. A unit dosage form including the solution and a vial or ampule comprising the unit dosage form are also described.

This invention relates to a stable injectable solution containing pamidronate, preferably made from pamidronic acid.

Pamidronic acid ((3 amino-1-hydroxypropylidene) bisphosphonic acid) can be used to produce pamidronate disodium which is a therapeutic active agent used for the treatment of hypercalcaemia and is used in medication for the treatment of diseases such as osteoporosis and tumor osteolysis. Pamidronic acid is practically insoluble in water, while the salts thereof are freely soluble. An injection solution of pamidronate can be prepared from pamidronic acid by adding sodium hydroxide into a suspension of pamidronic acid in water, preferably in 1:2 molar ratio. In a prepared solution, the active agent will present as pamidronate anions in the near neutral pH range, eg., 5-9 and is generally called pamidronate. A convenient method for administering this active agent is by intravenous infusion into the bloodstream of a patient to be treated. This invention provides a stable and pre-prepared injectable solution of pamidronate ready to be diluted by a practitioner administering the product to the patient. This enables the product to be provided in a consistent quality and avoids the need for the practitioner to reconstitute the active agent at the time administration is required.

According to one aspect, the present invention provides a stable pharmaceutical product including a sealed container containing pamidronate in solution, the solution having a pH of between 5 and 8, the solution being free of organic acid buffer.

According to a further aspect, the present invention provides a pharmaceutical product including a container containing pamidronate ions in solution, the solution having a pH of between 5 and 8 being free of organic acid buffer, wherein the container consists of at least one component, and wherein at least one component is manufactured from glass; whose solution contact surface is pre-treated so as to reduce or lessen the extent to which impurities are leached from the glass.

In order to obtain adequate long term stability, appropriate containers must be used for the solution. Appropriate containers for this product include ampoules, vials, bottles, ready to use syringes and Shell Glass Vials. Glass has long been the material of choice for pharmaceutical products. However, it has been found that pamidronate solutions left in glass for extended periods display unacceptable levels of turbidity despite the good solubility and chemical stability of pamidronate. It is believed that the principal cause of this turbidity where glass containers are used is the leaching out from the glass of aluminium and/or other cations such as magnesium or calcium, depending upon the glass composition. Where glass containers are used it is necessary to treat the surface of the glass with an appropriate method to reduce or lessen the extent to which impurities leach from the glass. A preferred method is a siliconization process using a one percent silicone solution to wash the vials followed by double draining and heating to 310° C. for thirty minutes. Vials pretreated in this manner are available from the French vial manufacturer Saint-Gobain Desjonqueres.

Other vial pretreatment techniques include the use of a high purity SiO₂ barrier formed on the inside vial surface by a plasma-deposition process. The process involves microwave energy being applied to a silicon containing precursor in the presence of oxygen. A plasma forms and a SiO₂ layer is formed on the glass surface from the gas phase. Vials pretreated in this manner are available from Schott.

In addition to treating the surface of the glass, it is also recommended to use containers which are made from glass having a low aluminum content. Glass typically used for pharmaceutical vials has in the order of 5 percent aluminium oxide. In order to reduce the problem of aluminum ion leaching, glass with lower aluminium ion content is recommended.

Where the solution is stored in a stoppered vial, the stopper provides a potential source of contamination. Typical elastomeric stoppers are potentially a source of calcium, zinc and magnesium ions which can react with pamidronate to form insoluble matter. In order to reduce the possibility of contamination, stoppers with low levels of these and other potential contaminants are to be used, preferably coated to form an inert barrier. An example of an appropriate stopper is the Daikyo D777-1 stopper. Daikyo D777-3 stoppers may also be suitable. Preferably the stopper has a low calcium, magnesium and ash content and is coated on the contact surface (being the surface of the stopper which when placed in a vial is exposed to the contents of the vial) with a fluorinated resin such as tetrafluoroethylene polymer, trifluorochloroethylene polymer, tetrafluoroethylene-hexafluoropropylene copolymer, fluorovinyldene polymer, vinylidene fluoride polymer, vinyl fluoride polymer, tetrafluoroethylene-ethylene copolymer, ethylene-tetrafluoroethylene copolymer, and perfluoroalkoxy polymer. In another embodiment of the present invention, the stopper is coated with a fluorinated resin selected from a group consisting of tetrafluoroethylene polymer, trifluorochloroethylene polymer, tetrafluoroethylene-hexafluoropropylene copolymer, vinylidene fluoride polymer, vinyl fluoride polymer, and tetrafluoroethylene-ethylene copolymer. For example, the stopper can be a FLUROTEC™ stopper manufactured and distributed by the Daikyo/Pharma-Gummi/West Group.

Vials may be constructed from any suitable other materials in addition to glass, such as polyethylene, polypropylene and polymethylpentene. For example, the vial could be constructed from CZ resin as manufactured by Daikyo/West.

The solution includes a suitable source of the active agent pamidronate ions. This includes pamidronic acid or any therapeutically acceptable salt thereof such as the disodium salt. The concentration of pamidronate is not critical, but will normally be in the order of 3-15 mg/mL.

It is known that the level of turbidity of pamidronate solution is affected by the pH of the solution and that the level of turbidity decreases with increased acidity. However, it is preferred to have a product within the biological range i.e. of between about 5 and 8, to reduce the incidence of potential adverse reactions relating to acidic solutions. Surprisingly it has been found that a stable solution can be produced having a pH of 5 - 8. A pH level in the order of 6.5 is preferred. At pH levels below about 5 there is a risk of producing venous type irritations and other unwanted side effects. pH levels above about 8 give rise to generally unacceptable levels of turbidity.

A solution of one percent pamidronate disodium in distilled water has a pH of approximately 8.3. In order to lower the pH a suitable acid is used. Suitable acids include any inorganic acid, such as hydrochloric or phosphoric acid. Hydrochloric acid is preferred.

As the person skilled in the art will appreciate, other standard components, such as mannitol and sodium chloride may be included in the solution, as desired.

Another possible approach to combating the problem of reaction between the active substances of glass is the use of buffers such as organic acids or polyethylene glycols. Whilst the use of such buffers may assist, it is generally preferable to minimize the number of additional constituents of any injectable material and the current invention provides a formulation without such buffers.

The present invention provides for a pamidronate solution having acceptable stability for up to at least eighteen months at room temperature.

Surprisingly, the inventors have found that it is possible to formulate a stable pamidronate solution which is neither highly acidic nor which involves the use of buffer systems. The inventors have found that solutions of pamidronate of relatively neutral pH values do exhibit satisfactory stability provided appropriate containers are used.

### EXAMPLE 1

In this example the product solution was composed of the following:

| | |
|---|---|
| pamidronic acid | 2.53 mg |
| mannitol | 47.0 mg |
| sodium hydroxide | 0.43 mg |
| pH | qs to 6.3-6.7 using 1.0 N sodium hydroxide |
| WFI | qs to 1.0 mL |

The solution was formulated using standard manufacturing processes and filled into 10 mL siliconised, low aluminium, type 1 glass vials, supplied by SGD. Each vial was enclosed by a 20 mm, S10-F451, D777-1, B2-40, Fluorotec stopper supplied by Daikyo/West.

Table 1 shows the test results measured over a 18 month period while being stored inverted at 25° C., relative humidity (RH) 60%.

TABLE 1

| | Initial (0 Months) | 6 months | 12 months | 18 months |
|---|---|---|---|---|
| Appearance | N | N | N | N |
| Potency | 97.5% | 99.5% | 100.8% | 99.9% |
| pH | 6.4 | 6.2 | 6.4 | 6.4 |
| Metal ions | | | | |
| silicon ppm | 0.23 | | 2.2 | |
| calcium ppm | <0.04 | | <0.04 | |
| aluminium ppm | <0.04 | | 0.1 | |

N - Clear colorless solution, free from visible particles.

### EXAMPLE 2

In this example the product solution was composed of the following:

| | |
|---|---|
| pamidronic acid | 7.58 mg |
| mannitol | 37.5 mg |
| sodium hydroxide | 1.29 mg |
| pH | qs to 6.3-6.7 using 1.0 N sodium hydroxide |
| WFI | qs to 1.0 mL |

The solution was formulated using standard manufacturing processes and filled into 10 mL siliconised, low aluminium, type 1 glass vials, supplied by SGD. Each vial was enclosed by a 20 mm, S10-F451, D777- 1, B2-40, Fluorotec stopper supplied by Daikyo/West.

Table 2 shows the test results measured over a 18 month period while being stored inverted at 25° C., relative humidity (RH) 60%.

TABLE 2

| | Initial (0 Months) | 6 months | 12 months | 18 months |
|---|---|---|---|---|
| Appearance | N | N | N | N |
| Potency | 105.1% | 106.0% | 105.9% | 107.2% |
| pH | 6.4 | 6.2 | 6.4 | 6.3 |
| Metal ions | | | | |
| silicon ppm | .4 | | 5.9 | |
| calcium ppm | 0.06 | | 0.1 | |
| aluminium ppm | 0.04 | | .29 | |

N - Clear colourless solution, free from visible particles.

### EXAMPLE 3

In this example the product solution was composed of the following:

| | |
|---|---|
| pamidronic acid | 2.53 mg |
| mannitol | 47.0 mg |
| sodium hydroxide | 0.86 mg |
| pH | qs to 6.3-6.7 using 1.0 N sodium hydroxide or |
| | 1.0 N phosphoric acid. |
| WFI | qs to 1.0 mL |

The solution was formulated using standard manufacturing processes and filled, into 10 mL siliconised, low aluminium, type 1 glass vials, supplied by SGD. Each vial was enclosed by a 20 mm, S10-F451, D777-1, B2-40, Fluorotec stopper supplied by Daikyo/West.

Table 3 shows the test results measured over a 12 month period while being stored inverted at 25° C., relative humidity (RH) 60%.

TABLE 3

| | Initial (0 Months) | 6 months | 12 months |
|---|---|---|---|
| Appearance | N | N | N |
| Potency | 103.6% | 103.5% | 104.0% |
| pH | 6.5 | 6.4 | 6.5 |
| Metal ions | | | |
| silicon ppm | 0.31 | 0.2 | 0.47 |
| calcium ppm | 0.06 | <0.04 | <0.04 |
| aluminium ppm | 0.17 | <0.04 | <0.04 |

N - Clear colorless solution, free from visible particles.

### EXAMPLE 4

In this example the product solution was composed of the following:

| | |
|---|---|
| pamidronic acid | 7.58 mg |
| mannitol | 37.5 mg |
| sodium hydroxide | 2.58 mg |
| pH | qs to 6.3-6.7 using 1.0 N sodium hydroxide or |
| | 1.0 N phosphoric acid. |
| WFI | qs to 1.0 mL |

The solution was formulated using standard manufacturing processes and filled, into 10 mL siliconised, low aluminium, type 1 glass vials, supplied by SGD. Each vial was enclosed by a 20 mm, S10-F451, D777-1, B2-40, Fluorotec stopper supplied by Daikyo/West.

Table 4 shows the test results measured over a 12 month period while being stored inverted at 25° C., relative humidity (RH) 60%.

TABLE 4

| | Initial (0 Months) | 6 months | 12 months |
|---|---|---|---|
| Appearance | N | N | N |
| Potency | 98.9% | 99.2% | 100.0% |
| pH | 6.5 | 6.4 | 6.5 |
| Metal ions | | | |
| silicon ppm | 0.29 | 0.3 | 0.65 |
| calcium ppm | 0.18 | 0.10 | 0.13 |
| aluminium ppm | 0.12 | <0.04 | 0.07 |

N - Clear colorless solution, free from visible particles.

It is understood that various modifications, alternatives and/or additions may be made to the product specifically described herein without departing from the spirit and ambit of the invention.

1. A method for preparing a therapeutic aqueous disodium pamidronate solution comprising:
(a) preparing a slurry of pamidronic acid in water, and
(b) combining aqueous sodium hydroxide with said slurry in an about 2:1 molar ratio of sodium hydroxide to pamidronic acid; to yield a solution of disodium pamidronate having visual clarity and a pH of about 6.5;
(c) packaging said solution in a plurality of sealed containers to yield a plurality of liquid unit dosage forms of pamidronate.

2. The method of claim 1 wherein the slurry includes an effective stablizing amount of mannitol.

3. A unit dosage form comprising a disodium pamidronte solution prepared by the method of claim 1.

4. A unit dosage form comprising a disodium pamidronate solution prepared by the method of claim 2.

5. A vial or ampule comprising a unit dosage form of the solution of claim 3.

6. A vial or ampule comprising a unit dosage form of the solution of claim 4.

7. The vial or ampule of claim 5 wherein the solution is packaged under an inert atmosphere.

8. The dosage form of claim 3 wherein the container is free of CA⁺² that can be sequestered by the disodium pamidronate.

9. The dosage form of claim 8 wherein the container is a plastic vial or ampule.

10. The dosage form of claim 8 wherein the container is a glass vial or ampule.
